(19) United States
(12) Patent Application Publication
(10) Pub. No.: US 2003/0228349 A1
(43) Pub. Date: Dec. 11, 2003

(54) Method for preparing an enhanced digestibility and stool index modulating composition for an animal

(76) Inventors: Bhajmohan Singh, Saint Louis, MO (US); Brigitte Martineau, Saint Louis, MO (US)

Correspondence Address: John S. Beulick, Armstrong Teasdale LLP, Suite 2600, One Metropolitan Square, St. Louis, MO, 63102, US

(21) Appl. No.: 10/167,188

(22) Filed: Jun. 11, 2002

(51) Int. Cl. A61K 47/00; A23K 1/165; A23K 1/17; C12P 21/06

(52) U.S. Cl. 424/442; 435/68.1

(57) ABSTRACT

A method for increasing the diet digestibility of food for an animal and modulating the firmness of fecal droppings (or stool index or excreta) of an animal which comprises conditioning an aqueous meat slurry comprising fat and meat (protein) by homogenizing and emulsifying the fat and meat ingredients in water, treating the conditioned slurry with an effective amount of an enzyme composition under conditions of pH and temperature for a time effective to cause hydrolysis resulting in the production of an enhanced digestibility and stool index modulating composition and utilizing this composition as an animal food or incorporating this stool index improving composition into an animal food, in an amount effective to modulate the stool index of animal droppings, and increase the food digestibility.

### BACKGROUND OF THE INVENTION

This invention relates generally to animal foods and more particularly to methods for producing animal foods having enhanced digestibility and/or animal stool index modulating properties.

Pet owners desire to take very good care of their pets as their pets are highly valued. For a long time, palatability and nutritional value of animal foods have been highly desired particularly among such pet owners. So over the years there has been an ongoing focus to improve methods and compositions, which enhance the palatability of animal foods while maintaining desired nutritional beneficial properties of the animal food.

Meat slurries are known and are employed to make a claim that pet foods contain “real meat”. These meat slurries are made with one or many kinds of meats, fats and other additives and incorporated in or used on pet foods. Besides the claim of “contains real meat” which is desired for super-premium category customers, meat slurries make pet foods more palatable than those pet foods which are made with rendered meat meals without “real meat.”

Enzyme treatment of meat slurries has been used in the pet industry to make highly palatable “digests” or coatings, which are then topically applied, to finished pet foods to increase the palatability of the foods. Many commercial vendors make “digests” and make them available to pet food manufacturers in spray-dried or concentrated liquid forms to increase the palatability of pet foods.

Hydrolysis is a class of reactions facilitated by enzymes that results in the breakdown of large molecules such as proteins and fats (hydrolytic reactions). Enzymatic hydrolysis of the proteins and fats obtained from food is a necessary part of the normal digestive system of animals and is helped by the naturally occurring enzymes in an animal body.

Proteolysis is the breakdown of proteins. Proteolysis can occur as a result of proteinases (enzymes used in the breakdown) in the food itself, e.g. autolytic reactions in meat, or with microbial proteinases, such as with the addition of microorganisms.

There are many classes of enzymes that facilitate hydrolytic reactions in proteins. For example there are exopeptidases that cleave amino acids or dipeptides in a stepwise fashion from one or the other end of the protein molecule. There are endopeptidases (or proteinases that hydrolyze the linkage within the peptide chain. Proteolytic enzymes that facilitate enzymatic hydrolysis of proteins can be found in any general food chemistry textbook such as one by H. D. Belitz and Grosch, Food Chemistry, 1987, ISBN 0-387-15049-3.

Similarly, fat hydrolysis is a breakdown of the fats and fatty acids. Lipases are used primarily for that purpose, and there are many classes of lipases used in the food industry.

Although the development of such palatable foods having adequate nutritional value continues, at times the animals' stool is less firm and the volume larger than that desired. Pet owners, breeders and veterinarians want a smaller, firmer stool (feces) that can be easily picked up. The volume of stool clean up is very important to an animal breeder or kennel operator who typically has a large number of animals. Thus, it is desired to have a method to provide firmer animal droppings of lower volume. Increased digestibility of food is related to a smaller stool volume and thus it is also highly desired to enhance the digestibility of food.

### BRIEF SUMMARY OF THE INVENTION

In one aspect, the invention comprises a method for modulating the stool index of droppings from an animal which comprises preparing an aqueous meat slurry comprising fat and meat, conditioning the slurry by homogenizing and emulsifying the fat and meat slurry in water, treating the conditioned meat slurry with an effective amount of an enzyme composition under conditions of pH and temperature for a time effective to cause a hydrolysis reaction resulting in the preparation (production) of a stool index modulating composition, and utilizing the stool index composition as an animal food or incorporating the stool index composition into an animal food, in an amount effective to modulate the stool index of an animal. In an embodiment, the method additionally comprises heating the stool index modulating composition to a temperature and for a time effective to substantially inactivate the enzyme(s) in that composition.

In another embodiment the invention comprises a method for increasing the diet digestibility of an animal food which comprises preparing an aqueous meat fat slurry comprising fat and protein ingredients by homogenizing and emulsifying said ingredients in water to form a conditioned slurry, treating the conditioned meat fat slurry with an enzyme composition under conditions of pH and temperature for a time effective to cause a hydrolysis reaction resulting in the production of a food digestibility enhancing composition, and utilizing the food digestibility composition as an animal food or incorporating the food digestibility enhancing composition in an animal food, in an amount effective to enhance the digestibility of the food. In an embodiment, the method additionally comprises heating the food digestibility enhancing composition to a temperature and for a time effective to inactivate the enzyme following the hydrolysis reaction.

In another embodiment, the invention comprises a pet food composition useful for modulating the stool index of droppings of an animal, said composition comprising an enzyme conditioned meat fat composition used with a food in an amount effective to modulate the stool index of an animal.

In another embodiment, the invention comprises a pet food composition useful for increasing the digestibility of animal food wherein the composition comprises an enzyme conditioned meat fat composition in an amount which when combined with an animal food is effective to enhance the digestibility of the animal food.

### DETAILED DESCRIPTION OF THE INVENTION

In accordance with one embodiment of the invention, a stool index modulating composition is prepared by admixing a meat and fat aqueous slurry with an active enzyme composition (to cause an enzymatic hydrolysis reaction), optionally inactivating the enzyme(s) with an applied heat treatment (increased temperature), combining the stool index modulating composition with other desired animal food additive(s) and extrusion cooking the composition to prepare a final product. Following extrusion, product pieces are spread out on a metal grid that flows into a dryer where the product is dried. The product then may have coatings applied before being packaged for shipment. The final product possesses an added functionality of being able to therapeutically modulate the animal stool to produce lower volume firmer animal stools and increased digestibility of food when provided to the animal as an animal feed or animal feed supplement.

As employed herein, the term “animal” includes carnivores (cats) and omnivores such as dogs of all breeds.

As employed herein, the term “modulate” includes a functional effect such as control and includes effects such as change, maintain, increase and decrease.

As employed herein, the term “therapeutically modulate” means the administration of an appropriate dosage to an animal as by a ration fed to the animal which provides for a resulting successful efficacious modulation in the stool volume and/or firmness of the stool or increase in the diet digestibility in a manner which is beneficial to the animal owner and animal.

As employed herein, the term “meat” includes animal tissue or rendered animal such as parts of cattle, pig, sheep, deer, elk, pork, buffalo, poultry (chicken, duck, goose and ostrich), meat derivatives and fish. Preferably non-rendered real animal (non-human) tissue is employed. It is understood that the “meat” comprises proteins which are hydrolyzable by an enzyme(s) in the presence of water.

As employed herein, the term “enzyme” includes a molecule or substance which has the capability to bring about a chemical transformation and includes proteins and proteolytic enzymes which has the capability to bring about the hydrolysis of a peptide bond or the hydrolysis of an ester bond in the presence of water. Enzyme includes mixtures of enzymes and includes lipases and proteases. A discussion of enzymes is found in BIOCHEMISTRY, Third Edition, Lubert Stryer, W. H. Freeman and Company, NY, 1988, Chapter 8, pages 177-200.

As employed herein, the terms “feces”, “fecal matter”, “stool” include the solid or semi-solid waste products of animal digestion and includes excreta and droppings.

As employed herein, the term “treatment” includes placing in contact with such as by placing an enzyme(s) in sufficient proximity or contact with a meat or fish to cause a hydrolysis reaction of the protein therein in the presence of water.

As employed herein, the term “conditioning” includes homogenization and emulsification.

As employed herein, the terms “therapeutic” and “effective” mean a dosage level to an animal as in a ration, equal to or greater than an amount which is minimally beneficial to the animal and that provides for a firmer and lower volume animal stool as compared to a stool produced by the animal without having taken (as by eating) a composition of this invention, but which is less than an amount which would cause discomfort to the animal in regard to its stool movements and necessitate a medical or manual disimpaction of the bowel as the animal bowel becomes firmer and of lower volume as a result of having taken a composition of this invention. The appropriate effective dosage for animals will be apparent after reading this specification including the examples provided herein.

As employed herein, the terms “therapeutic dosage,” “therapeutic amount,” and “effective amount” mean that the dosage and the amount provided to the animal is beneficial to the animal in a therapeutic manner.

As employed herein, the term “taken” includes eating, ingestion and administration by usual and appropriate means.

As used herein the term “utilizing” includes used with (as a supplemental food for example a supplemental ration to a food or used as a stand alone ration), or used on, applied to, and used as an animal food itself. The term “incorporated” includes using as an additive with an animal food, used in an animal food or in association with an animal food.

At the start of the preparation process, an aqueous meat fat slurry is prepared typically by heating frozen slabs of meat which are ground and then intimately admixed with fat and water to form a homogenized and emulsified aqueous composition (suspension) of solids and liquid(s). The resulting meat/fat/water composition is thoroughly and intimately mixed to prepare a homogeneous meat/fat/water slurry. Typically, the size of the meat in the aqueous meat slurry is less than about ¼ inch in size.

Meat fat water slurry preparation is an important aspect of this invention and is carried out by mixing thoroughly and intimately the meat-fat water slurry in order to make an aqueous homogeneous slurry that will be better suited for treatment with an enzyme(s). If desired, homogenization of the slurry can be achieved by recirculating through a slurry pump, which acts on the meat and fat to reduce particle size in the slurry and thoroughly mix the various components of the slurry. The objective of homogenization is to reduce the size of meat and fat globules in order to distribute them substantially equally throughout and produce a uniform emulsion.

Good emulsification of the mixture is desired to enhance a hydrolysis reaction. Good emulsification enhances effective contacting of the enzyme with the meat and fat so that a desired enzyme reaction process will occur.

Various fats can be employed to prepare the slurry composition. Physically the fat can normally be a solid or a plastic fat; however, the fat should be heated before homogenization for best slurry homogenization results. Mixtures of fats can be employed if desired.

The amounts of water and fat are not considered critical but are in the range from about 0% to about 80% and from about 0% to about 25%; sufficient, preferably vigorous agitation is employed to achieve desired emulsification. The amount of agitation is sufficient for an effective time to accomplish emulsification but generally not to diminish or destroy activity of the enzyme composition.

Fat(s) employed in the emulsification are preferably those fats which will hydrolyze in the presence of protease and lipase enzymes under favorable hydrolysis conditions so as to enable conditioning and hydrolysis of the meat fat slurry.

Fats used are typically triglycerides, triacylglycorals, mixtures thereof and/or derivatives thereof and are preferably animal fats. Useful animal fats include those fats which naturally occur in meats, including tallow, chicken fat, butter oil, shortening and lard. Generally, animal fats such as beef tallow are preferred fats. In an exemplary embodiment, those fats which are palatable or which by processing result in a food composition, which is palatable to an animal, are preferred. It is preferred to use real fat or fats.

If desired, emulsification can be accomplished by use of effective mechanical agitation such as a Tekmar emulsifier wherein meat, fat and water are emulsified in the Tekmar. During emulsification, sufficient agitation is supplied for a time effective to prepare an emulsion.

If desired, pH modification of the slurry may be carried out. Agents which modify pH may be added such as an acid (to decrease pH) or a base (to increase pH) to predetermined or selected pH levels.

The molecular weight distribution (weight average) of the meat/fat/water slurry (untreated with enzyme) is in the range from about 25,000 daltons to about 100,000 daltons.

After the meat/fat/water slurry is prepared and conditioned, the enzyme is admixed with the slurry and stirring is maintained for the enzyme reaction duration to maintain a sufficiently desirable high rate of hydrolysis of meat and fat. The reaction and its conditions will be monitored and continued for a time effective to sustain and complete the enzymatic reaction desired resulting in the preparation of a stool index modulating composition. If desired, the time, temperature and pH may be predetermined which are selected to obtain a selected degree of hydrolysis of the meat fat slurry composition.

If desired, an emulsifier can be used to stabilize the fat in water emulsion. In that regard, ingredients useful as stabilizers indude teraglycerol monostearate, polyoxyethylene sorbitan monostearate, sodium stearoyl-2-lactylate, polyoxyethylene sorbitan tristearate, sodium oleate, mixtures thereof and the like.

It is preferable to add the enzyme to the slurry although the slurry may added to the enzyme, such as in the case of immobilized enzymes.

The enzyme(s) employed may be obtained or derived from any suitable active enzyme source or commercial supplier as long as the enzyme is sufficiently active and contains effective amounts of protease or protease and lipase when used at concentrations which effectively enable a hydrolysis reaction and which will not adversely effect the palatability of animal food. One commercial supplier of enzymes is Novozymes of Franklin, N.C., 27525.

An individual enzyme or a mixture of enzymes may be employed. A preferred protease is a proteolytic enzyme in a concentrate from about 0.05% to about 2.0% w.w. of the meat slurry, depending on the activity of the enzyme and the time, temperature, pH conditions employed in the reaction.

Useful proteases include proteolytic enzymes such as Alcalase 2.4 L of Novozymes Biotech, Inc, 1445 Drew Avenue, Davis, Calif., 95616

The protease may be used with a useful lipase(s), which includes lipolytic enzymes such as Palatase or Lipase of Novozymes Company.

The concentration of the enzyme mixture is not critical as long as the enzyme(s) is present in amounts effective, under the desired favorable enzymatic conditioning reaction conditions to cause and result in the production of a stool index modulating composition and/or a food digestibility increasing composition.

If desired, the enzyme composition can be admixed with the meat and fat aqueous composition before or after the fat is emulsified with water, however addition of the enzyme to the composition is preferred after emulsification. Tap water may be used as the water source if desired.

In order to initiate and carry out the enzyme reaction, the emulsified composition is heated to a temperature in the range from about 120.° F. to about 170° F. and preferably from about 140° F. to about 150° F. Heating may be accomplished by adding steam to a vessel, such as a kettle, holding the emulsified composition. The desired enzyme reaction temperature is that temperature at which effective conditioning reaction can occur and be completed in a desired time without causing a rapid loss in activity in the enzyme reaction. It is recognized that increasing the temperature of the composition may enhance the reaction. However, the temperature is preferred to be maintained for at least some time at less than that which would totally inactive the enzyme.

Control of the pH of the emulsified composition is typically needed or employed so that hydrolysis reaction occurs preferably in optimized fashion. Typically, the pH of the emulsified composition is in the range from about 4 to about 11 and preferably from about 6 to about 8 although greater and lower values of pH may be used according to conditions of reaction, types of meat and enzyme use and other factors. The pH of the composition is typically monitored so that adjustments may be timely made if needed.

Compositions useful to adjust the pH include any composition, which will after addition to the composition provide a desired pH. The amount of such composition is such to adjust the pH of the emulsified composition to the desired pH range.

The duration time of the hydrolysis reaction is that elapsed time from the start of the hydrolysis conditioning reaction to the termination thereof which is a sufficient elapsed time to carry out the desired reaction and is in the range from about 20 minutes to about 120 minutes, and preferably from about 30 to about 60 minutes, although from about 30 minutes to about 45 minutes is most preferred.

If desired, the degree of hydrolysis may be monitored as by sampling of the process composition and performing an analysis thereon, at various time intervals throughout the process. When a desired amount of hydrolysis has occurred (resulting in the appropriate or a desired ratio of (free) amino acids and peptides), or a desired molecular weight distribution of the slurry, or a desired viscosity of the slurry or when the hydrolysis is complete or sufficiently complete the enzyme activity may be stopped and the enzyme inactivated usually by thermal means. Typically the degree of hydrolysis is measured by running a Size Exclusion Chromograph (SEC) of the slurry. SEC is also known as gel filtration or gel permeation (GPC). This method uses porous particles to separate biological molecules providing molecular weights and molecular weight distributions of polymers. See: http://www.chem.vt.chem-ed/set/lc/size-exc.html.)

Typically the degree of hydrolysis (DH) desired is such that in this system, a treated meat fat water slurry (product of the hydrolysis reaction) has an molecular weight distribution (weight average) in the range from about 5,000 Daltons to about 30,000 Daltons. A molecular weight distribution (weight average) of about 11,000 Daltons to about 13,000 Daltons is preferred for a treated meat fat water slurry. The unhydrolyzed meat fat water slurry will have a molecular weight distribution (weight average) in the range of about 25,000 Daltons to about 100,000 Daltons.

Following the hydrolysis reaction, the stool index improving composition is optionally heated to a (higher) temperature and for a time effective which is sufficient to inactivate the enzyme(s).

Typically the temperature during enzyme inactivation is in the range from about 160° F. to about 190° F. and preferably from about 170° F. to about 180° F. The increase in temperature over the temperature employed in the hydrolysis reaction deactivates the enzyme. Those of skill in the art will recognize that greater or lesser temperatures or times may be employed which will be a function of parameters such as meat, fat, enzymes and the time employed and other operating variables. Further processing steps such as extrusion (a high temperature process) can serve to deactivate the enzyme. If, however, live active enzymes are desired in the food product, then the deactivation step does not need to be carried out.

The duration of the deactivation of the enzyme is that elapsed time effective to inactivate the enzyme composition. This time is generally in the range from about 5 minutes. to about 20 minutes and preferably from about 10 minutes to about 15 minutes. Greater or lesser times may be employed which will be a function of operating parameters such as temperature, meat, fat, enzymes and other operating variables.

After the enzymes are deactivated or made inactive, optimal compatible ingredients may be admixed with the stool index composition to form an intermediate animal food composition or supplement.

Such compatible ingredients typically include but are not limited to fibers, medicines, fillers, diluents, prebiotics, probiotics, and mixtures thereof and the like.

The amount of animal stool index modulating composition used in, with, as or within animal food composition is a therapeutic amount which in the animal food provides or brings about (produces) a desired amount of increase, decrease or change in the stool index of the animal eating that food, provides a lower volume of stool and increases the digestibility of the food. This amount will vary for different animals such as for cats and dogs and for the size and age of the animal among other factors.

The intermediate animal food composition will preferably have a thin viscous or thick consistency that is flowable and can be metered or measured. Various food ingredients such as raw starch constitutes, protein binders together with solutes, edifying and characterizing ingredients such as food dyes and flavors or flavor pre-cursors as well as antimycotics and antibiotics may be admixed intimately to prepare a finished slurry suitable for inclusion into a desired animal food product.

In one embodiment, the finished slurry is fed to an extrusion feed zone. If desired, a dry base may be added to the composition prior to or at the time of entering the extrusion feed zone or is first mixed with the slurry prior to extrusion of dry meat base and aqueous meat slurry.

Prior to entering the extruder, the food slurry will be maintained at an elevated temperature generally in the range from about 140° F. to about 200° F. and preferably from about 160° F. to about 180° F.

The food slurry will be intimately mixed under conditions effective to achieve effective mixing; including rigorous agitation, whereby suitable dry mix ingredients previously recited will be substantially well mixed therewith.

The mixing can be suitably carried out in suitable mechanical mixture such as those carried out by mixing in an agitated conditioner for extrusion.

In the extrusion process, the input ingredients are further cooked and extruded. Cooking extrusion can be carried out in a continuous or batch extrusion cooker comprising a screw fed, steam jacketed or suitable heated chamber wherein the food slurry and admixed dry solids are effectively combined to produce an extrudate comprising a stool index modulating composition. Within the extruder, mechanical energy is imparted to the composition being extruded by means of rollers or helical screws which are driven by gears connected to an external mechanical or electric driving source such as a motor. This significant and substantial energy input to the composition results in a temperature increase in the material being extruded. If desired, external additional heat may be applied to the composition by heating coils located on the extruder, etc. The composition being extruded is generally masticated and is thoroughly and intimately mixed by the action of the extruder screws upon the composition.

The extruder screws are generally designed and configured to provide high mechanical energy input to the composition being extruded as they are rotated. This heat and mechanical energy input to the composition in the extruder results in a masticated or substantially masticated composition in the extruder. The screw configuration and rotation is such that the masticated composition is moved forward from the feed zone entry toward a die cap extruder exit. In the process of converting input ingredients into dog food, there is an infusion of steam and moisture at high temperature into the extruder feed. As a result the food is further cooked and formed.

The duration time of cooking extrusion is an elapsed time effective to accomplish desired further cooking and extrusion of the composition. The extruder product which is an extrudate will generally be hot but which can be mechanically cut and suitably fashioned to any desired size and shape preferably of a size and shape convenient for eating by an animal such as a dog or a cat.

The extrudate typically passes through a die, such as a die cap, which have any desired suitable shape in it such as a cylinder, star, square or round shape. The particular shape selected is generally for aesthetic purposes and the shape is not critical. The size of the extrudate is generally small for ease of animal consumption as by ingestion by animals for example as kibbles. Generally the extrudate is continuously mechanically subdivided to thicknesses varying from about ⅛ inch to about one inch in length and diameter. If desired, the extrudate may be in pellet form for ease of handling and packaging. Typically such pellets are round or substantially round for ease of handling and oral consumption by an animal.

The extruded pellets are cooled to room temperature by passing the pellets over a cooling screen or mechanical equivalent whereby the temperature of the extruded pellets is lowered. During cooling the pellets take their final shape.

The pellets may be coated in whole or part with a palatability enhancing composition. If desired, the pellets are coated with a fat composition or digest which is palatable to an animal. Typically such coating is carried out by spray coating the pellet(s). If spray coating is used, then spray-coated product is generally allowed to set so that the coating is somewhat tackified before further processing or handling of the coated product. It is preferred that the coating be uniform on the pellets.

After cooling, the product is at room temperature, and it is placed on a system for filling the pellets into a consumer container such as a pouch. The product may be placed on a feeding vibrator for example and conveyed to a weigh scale system wherein weighed amounts of product are automatically loaded into individual pouches. Typically, such pouches are polyethylene coated aluminum foils, which are suitable for distribution through a warehouse system and ultimately are provided to consumers for purchase. The pouches are typically packaged. The packages normally are conveyored to a palletizer, which assembles packages in-groups on pallets, which are then shrink-wrapped for shipping to distribution points.

In an alternative embodiment, the enzymatically treated composition of this invention is produced as a product in cans. In a further alternative embodiment, the enzymatically treated composition of this invention is produced as a treat.

After purchase, the animal owner opens the pouches as desired and the product containing the stool index modulating composition is fed to an animal as part of the animal's ration or feed. After the animal eats the ration or food, the product is thus made available to the gastrointestinal tract of the animal for digestion and absorption via the digestion process into the animal's biosystem so that it is made bioavailable to the animal.

It is understood that the amount of stool modulating composition provided to the gastrointestinal tract of the animal is that therapeutic amount or dosage, including dosage level and rate, which provides a lower-volume firmer stool. The stool modulating composition may be combined or formed in any pharmacological acceptable composition if desired and administered to the animal as by oral administration (eating). If desired the stool modulating composition and digestibility enhancing composition may be provided to an animal as a topical or digest applied to a food ration provided to the animal.

It is understood that the benefits of the invention, such as firmer stool and lower stool output, will be visually apparent. The amount of digestibility enhancing composition provided to an animal is an amount which provides for a suitable enhancement of the digestibility of the food to an animal.

Some animals have sensitive stomachs for one reason or another. The stool modulating composition of this invention provides specially broken-down proteins for the sensitive stomach and easier to digest real-meat proteins. The higher digestible food helps during periods of stress; specially processed proteins help during recovery, as does a higher digestible formula. If desired this food digestibility enhancing composition can be a component of a specially or customized designed food for young and older animals such as senior animals.

Generally animals have special-need phases where, for example, the animal is recovering from an illness. If desired, this composition can be utilized as a component for foods for an animal in a recovery phase. Additionally animals undergo stress at times as for example when animals are in close proximity to one another.

Additionally the composition of this invention may be utilized in or as a formula for a high stress period for an animal and for puppy and kitten formulas. The composition is also a limited antigen, real meat composition. Generally the composition is also a lowered antigen composition as result of having been treated with protease and/or lipase. The composition may also be used as a component in real meat biscuits for animals with sensitive stomachs, kennel and breeder animal foods and for multiple cat households where multiple cats cause anxiety or stress to each other. The process offers an additional benefit in that pathogens in any meats employed in the meat fat water slurry may be inactivated or substantially inactivated as a result of undergoing enzymatic reaction and/or subsequent heat treatment.

This invention further provides a way of providing needed nutrition rations for puppies/kittens in the form of “comfort proteins”. Such puppies/adults need meat proteins (for essential amino acids) but do not yet have fully matured digestive tracts to adequately digest those proteins.

Exemplary embodiments of the invention are described in the following examples. Other alternative embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

The methods used were as described in the examples below. Specific materials used in the work reported herein were as described herein, but those skilled in the art will recognize that many suitable alternative materials are readily available from commercial suppliers.

### EXAMPLES

### Example 1

This Example presents the results of an enzymatic hydrolysis of real meat prior to extrusion which was carried out to investigate its impact on pet food digestibility, firmness, pet food palatability and pet stool quality of formulations as follows.

Pet food digestibility tests assess the percentage of nutrients available for metabolic processing. Firmness is a gauge of the degree of rigidity of the pet stool.

A fecal scoring system for dogs and cats was used to determine the stool quality index. In this system, individual fecal events (or droppings) are visually scored according to published charts. Canine feces are scored on a five point linear increasing scale whereas cat feces are characterized on a four point linear increasing scale. The most loose/runny stools are at one end of the score (score=zero) and hard/firm stools are scored as 100 at the other end of the fecal scoring scale in an increasing numerical scale. A statistical number of dogs are on each ration and then a mixed-model analysis of variance is used to assess the effect of ration on the calculated stool quality index. Least squares means and standard errors of the fecal scores are calculated, and pairwise differences are evaluated with t-tests to determine statistical significant differences between stool quality scores.

In order to gauge dogs' taste preferences, palatability tests are conducted. In this test, dogs are given free choice of two foods side by side during two-day feeding trials, and the quantity consumed of each food by the dogs is statistically analyzed.

High (0.25% w.w) and low (0.075% w.w) dosages of a proteolytic enzyme (Alcalase 2.4L, Novozymes Company, North Carolina) and a treatment of proteolytic and lipolytic enzyme cocktail (Alcalase 2.4L 0.25% w.w with Lipozyme 0.005% w.w) were tested on both a dog food (with >15% real beef meat) and a cat food (with >15% real chicken meat) formulation as rations. The meats were obtained frozen and were heated to the optimal temperature (135° F.) prior to enzyme addition and kept for recommended time (30 min to 60 min). Degree of hydrolysis (DH) obtained was measured using a titration assay. Hydrolyzed meats were mixed with dry ingredients and extruded. A highly palatable digest coating was applied to the dry finished products as per current practice. Control rations were same formulation and processing conditions but lacked the enzyme treatment of meat. Effects on palatability were assessed using two bowl choice tests and digestion and pet stool quality was evaluated using a standard fourteen-day digestion test.

In this test, two-day palatability results for both dog and cat food showed not much significant difference to a slight improvement in palatability for the treatment ration compared to the control ration. Significant incremental palatability was probably not obtained due to application of existing highly palatable topical coatings to rations of both the control and treatment groups. However, it is possible that for pet food formulations that do not have highly palatable topical coatings applied, that hydrolysis of meat slurry content would yield significant improvements in palatability.

Significant improvements were observed in pet stool scores for both the dog and cat food formulations in the above tests for the treatment group over compared to control group. (As noted above, the pet stool firmness score is a number, this is an indicia of the firmness of the stool.) In dog digestion tests conducted on a mix of Siberian Huskies and English Setters, with six dogs total in each group, the pet stool firmness score increased from 72.17 to 78.65 at a significant level (p<0.05). In a cat digestion test conducted on six cats each per group, the cat pet stool scores increased from 81.87 to 91.56 but with higher variability within the test group.

### Example 2

In this Example another test was carried out to illustrate this invention. In all Examples, the treatment ration represents a ration of this invention whereas the control ration represents a ration known in the art.

Test Conditions:

Control Ration: A super-premium formula (available from Ralston Purina, St. Louis, Mo.) made with real beef slurry

Treatment Ration: A super-premium formula, with the beef slurry treated with enzymes.

Hydrolysis Conditions:

Enzyme/hydrolysis conditions: (Alkalase 2.4L, Novo Nordisk, at 0.25% w.w, and Lipozyme, Novo Nordisk, at 0.005% weight per unit weight (“w.w”) added to the beef/tallow slurry (90/10) at 135° F. for 90 minutes in a slurry tank. The temperature was raised to 160° F. with steam addition to the slurry tank to deactivate the enzymes and the treated slurry was transferred to a hold tank. From then on the process was similar to the process for the control group.

Result: A 14 day standard digestion test was performed for the two rations. Stool index was higher (78.7) for treated ration compared to control ration (72.2) at p<0.05.

### Example 3

Two additional tests were conducted with a higher number of animals to increase the statistical power of the test. (The statistical power of the test is calculated by noting the increased sample size (number of dogs), the effect size (which is the salience of the treatment compared to any noise in measurement), the alpha-level (which is chance). The higher the statistical power the greater the odds that an observer will see an effect when it occurs.)

This example comprised two digestion tests. In summary, test results done at a first facility with Siberian Husky puppies (6 months of age) show slurry hydrolysis had a significant beneficial effect on total, protein, fat, ash, and carbohydrate digestibility with higher values obtained with hydrolysis. Digestible energy and metabolizable energy tended (p<0.10) to be higher with hydrolysis.

In another second test done on adult dogs at a second facility with English setter adult dogs with the same rations, slurry hydrolysis also significantly increased total, protein, ash, calorie and carbohydrate digestibility but left unchanged the high digestible and metabolizable energy and stool scores observed in the control group. Thus, there was consistency in results obtained between the two tests done at different facilities and with different breeds. The degree of hydrolysis (DH) obtained for the hydrolyzed slurry samples was significant and reduced the weight-average mean molecular weight from 26,000 Daltons for control slurries to between 11,000 to 13,000 Daltons for the hydrolyzed slurry of the invention.

Additional Test Details:

Test 1 was designed to be run at the Alaska research facility on puppies and investigated the effect of both slurry hydrolysis and fine grind on diet digestibility and fecal scores. Test 2 was run at a second research facility in Missouri on adult dogs and only investigated the effect of slurry hydrolysis on digestibility and stool quality.

Test Ration Descriptions

R1:A super premium formula (available from Ralston Purina, St. Louis, Mo.) made with real chicken slurry

R2: The same super premium formula but with slurry hydrolysis (Alcalase 2.4L/0.125% and Palatase 0.005%)

R3: Same formula as Ration 1 but made with Fine Grind of dry meal

R4: Same ration as R2 (but with slurry hydrolysis and fine grind of dry meal)

All four rations (R1-R4) were used in Test 1. However only R1 and R2 were used in Test 2.

Test Animals and Experimental Design:

In Test 1, a cross-over design was selected to be run. Thirty-two puppies were divided into four groups. Two of the groups were twenty weeks of age and two were thirty-two weeks of age. Each dog was assigned to two of the four rations in the test in an incomplete block design. The rations were balanced as much as possible within groups of puppies, order of presentation and pairing with other rations. Three days of total fecal collection was performed after a two-week adaptation period to each ration.

Five dogs were removed from the study for non diet-related reasons after the first feeding period and one dog was added to the study for the second feeding period.

In Test 1, analysis of variance for a mixed effects model was used to test for the effect of slurry hydrolysis, fine grind and the interaction of those two variables on digestibility variables. The test period and dog groups were considered as random effects. Repeated observations on individual dogs due to the crossover design were taken into account. In Test 1, Siberian Husky puppies were used.

In Test 2, a standard digestion protocol was used except higher number of dogs (18 each) was used on each ration for higher statistical power. The higher numbers of dogs provide the capability to detect differences of 7.5 or higher in the pet stool scores between the two groups. English Setter and Siberian Husky adult dogs were used.

Test Processing Conditions:

Fine Grind Conditions:

Rations 1 and Ration 2 were made with dry meals ground to a module of fineness (MOF) value of 127.

Ration 3 and Ration 4 were made with dry meals ground to an MOF of 95.

Slurry Hydrolysis Conditions:

Enzyme dosage/conditions: 0.125% ww Alcalase 2.4L and a 0.005% w.w Palatase was added to the slurry batch. This worked out to be 363 grams Alcalase enzyme and 72 grams Palatase enzyme dosage per slurry batch of 600 lb meat/tallow plus 55 lb of steam/water.

The temperature of slurry hydrolysis was set to 135° F. and maintained for 1.5 hours, after enzyme addition.

Degree of Hydrolysis Obtained:

Samples were taken for molecular weight distribution as a measure of degree of hydrolysis achieved. The method used was Size Exclusion Chromotograph (SEC). Results show that the average molecular weight (weight average) was 26,000 Daltons for the control slurry ration, and which reduced to 13,000 Daltons and 11,000 Daltons for two samples submitted after the slurry was hydrolyzed and deactivated. The intrinsic viscosity decreased from 2.61 to 1.34 and 1.14 for the same two samples submitted.

Rations 1 through 4 were made by combining the respective slurries with fine or non-fine dry meals, extruding, drying and coating per standard protocol.

Test Results

In Test 1, slurry hydrolysis was found to have a significant effect on total, protein, fat, ash and carbohydrate digestibility. In all cases, digestibility values were lower for dogs fed rations without hydrolysis. Digestible energy and metabolizable energy tended (p<0.10) to be lower without hydrolysis. These results demonstrate the enchanced digestibility of a composition of the invention. Fine grind was found to have a significant affect on feed consumption but not on the digestibility variables. Feed consumption was greater without fine grind. Metabolizable energy tended (p<0.10) to be lower without fine grind. A significant interaction was found between slurry hydrolysis and fine grind for carbohydrate digestibility. Dogs fed a ration with slurry hydrolysis and without fine grind had significantly higher carbohydrate digestibility than the two groups fed rations without slurry hydrolysis. A significant interaction between slurry hydrolysis and fine grind was found for fecal pH. These results mentioned are tabulated in Attached Table 1.

TABLE 1
(Digestion Summary by Main Effects)

| | | Slurry hydrolysis | | | Fine Grind | | |
|---|---|---|---|---|---|---|---|
| | | Control | Treatment | p-value | Control | Treatment | p-value |
| Number started | | 30 | 30 | | 30 | 30 | |
| Initial average weight | kg | 36.7 | 36.4 | ns | 36.5 | 36.6 | ns |
| (32 dogs) | | | | | | | |
| Final average weight | kg | 38.3 | 38.9 | ns | 38.9 | 38.4 | ns |
| (19 dogs) | | | | | | | |
| Dry feed/animal/day | g | 310 | 315 | ns | 322 | 303 | 0.05 |
| Feed consumption/ | g/kg | 8.4 | 8.6 | ns | 8.8 | 8.2 | 0.05 |
| body weight | | | | | | | |
| Wet feces/animal/day | g | 201 | 191 | ns | 206 | 186 | ns |
| Feces dry matter | % | 30.1 | 28.6 | 0.10 | 28.9 | 29.8 | ns |
| Diet Moisture | % | 7.5 | 7.7 | | 7.2 | 8.0 | |
| Diet Protein | % | 33.4 | 32.6 | | 33.1 | 32.8 | |
| Diet Fat | % | 20.1 | 20.9 | | 20.5 | 20.5 | |
| Diet Fiber | % | 2.2 | 2.0 | | 2.1 | 2.1 | |
| Diet Ash | % | 6.3 | 6.0 | | 6.4 | 5.9 | |
| Diet Calories | C/g | 5.2 | 5.2 | | 5.2 | 5.2 | |
| Total digest | % | 80.6 | 82.9 | 0.01 | 81.7 | 81.9 | ns |
| Protein digest | % | 88.1 | 89.4 | 0.01 | 88.8 | 88.6 | ns |
| Fat digest | % | 92.3 | 93.5 | 0.05 | 92.9 | 92.9 | ns |
| Ash digest | % | 23.8 | 35.1 | 0.01 | 29.5 | 29.4 | ns |
| Calorie digest | % | 86.1 | 87.4 | ns | 86.6 | 86.9 | ns |
| Carbohydrate digest | % | 82.1 | 83.7 | 0.01 | 83.0 | 82.7 | ns |
| Digestible energy | C/kg | 4457 | 4532 | 0.10 | 4465 | 4524 | ns |
| Digestible energy | C/lb | 2023 | 2058 | 0.10 | 2027 | 2054 | ns |
| Metabolizable energy | Kcal/kg | 4093 | 4168 | 0.10 | 4095 | 4219 | 0.10 |
| Metabolizable energy | Kcal/lb | 1858 | 1892 | 0.10 | 1859 | 1890 | 0.10 |

Key to terms:
ns = not significant
Kg = kilogram
g = gram
C/g = calories per gram
p-value = measure of significance
Kcal = kilocalorie
lb = pound
Kcal/kg = kilocalorie per kilogram of food
g/kg = grams feed per kilogram body weight
c/kg = calories per kilogram

Table 2 following shows the digestion test summary for Test 2. Droppings index (measure of stool quality) was nearly perfect for both groups and was not significantly different. Total, protein, ash and caloric digestibility was higher for the hydrolyzed group at p<0.05 significance level. Carbohydrate digestibility was also higher at the p<0.01 significance level.

Thus, tests 1 and 2 results are consistent among the two panels of dogs tested. The significant increases in digestibility were obtained using slurry hydrolysis.

TABLE 2
(Digestion Summary by Treatment Groups).

| | Ration | 1 | 2 | P-value |
|---|---|---|---|---|
| Number started | | 18 | 18 | |
| Number removed | | 0 | 0 | |
| initial weight | kg | 26.5 | 25.3 | Ns |
| final weight | kg | 26.8 | 25.6 | Ns |
| dry feed/animal/day | g | 300.8 | 306.7 | Ns |
| feed consumption/body | | | | |
| weight | g/kg | 11.7 | 12.2 | Ns |
| wet feces/animal/day | g | 157.1 | 148.3 | Ns |
| feces dry matter | % | 35.6 | 34.6 | Ns |
| Droppings Index | Score | 97.2 | 95.5 | Ns |
| Diet Moisture | % | 7.3 | 8.5 | |
| Diet Protein | % | 33.1 | 32.8 | |
| Diet Fat | % | 20.7 | 20.2 | |
| Diet Fiber | % | 1.9 | 2.0 | |
| Diet Ash | % | 6.6 | 6.4 | |
| Diet Calories | C/g | 5.2 | 5.1 | |
| total digest | % | 81.0 | 83.3 | 0.05 |
| protein digest | % | 88.5 | 90.2 | 0.05 |
| fat digest | % | 94.2 | 94.6 | Ns |
| ash digest | % | 23.3 | 32.4 | 0.05 |
| calorie digest | % | 87.2 | 88.6 | 0.05 |
| Carbohydrate digest | % | 80.3 | 82.6 | 0.01 |
| Digestible energy | C/kg | 4498 | 4524 | Ns |
| Digestible energy | C/lb | 2042 | 2053 | Ns |
| Metabolizable energy | Kcal/kg | 4131 | 4154 | Ns |
| Metabolizable energy | Kcal/lb | 1876 | 1886 | Ns |
| Wherein c = calories | | | | |

An advantage of this stool index modulating composition is that a process is provided for enzymatic modification/conditioning of meat/fat slurry employed in animal food to provide a stool index composition which is employed as a functional ingredient or supplement in animal food systems to modulate the stool index of an animal and increase digestibility and palatability of animal food. As a further advantage, firmer animal droppings facilitate stool cleanup and reduce clean-up materials and supplies as does lower droppings volume.

While the invention has been described in terms of various specific embodiments, those skilled in the art will recognize that the invention can be practiced with modification within the spirit and scope of the claims.

What is claimed is:

1. A method for modulating the stool index of droppings of an animal which comprises admixing an aqueous meat fat slurry comprising fat and meat ingredients to form a conditioned slurry by homogenizing and emulsifying said ingredients in water, treating the slurry with an enzyme composition under conditions of pH and temperature for a time effective to cause a hydrolysis reaction resulting in the production of a stool index modulating composition, and utilizing the stool index composition as an animal food or incorporating the stool index improving composition in an animal food in an amount effective to modulate the stool index of droppings of the animal.

2. A method in accordance with claim 1 which additionally comprises heating the stool index modulating composition to a temperature and for a time effective to inactivate the enzyme following said hydrolysis reaction.

3. A method in accordance with claim 1 wherein said effective amount is a therapeutic amount.

4. A method in accordance with claim 1 wherein said method further comprises selecting one or more ingredients from beef, lamb, chicken, pork, mutton, fish and poultry.

5. A method in accordance with claim 1 wherein the animal is a dog.

6. A method in accordance with claim 1 wherein the animal is a cat.

7. A method in accordance with claim 1 wherein said modulating comprises increasing the firmness of animal stool droppings.

8. A method in accordance with claims 1 wherein the stool index is increased and the degree of hydrolysis achieved is such that the weight average of the molecular weight distribution of the hydrolyzed slurry is in the range from about 5,000 Daltons to about 30,000 Daltons.

9. A method in accordance with claim 1 wherein modulating comprises decreasing the volume of stool droppings.

10. A method in accordance with claim 1 wherein the enzyme composition comprises a protease enzyme.

11. A method in accordance with claim 1 wherein the amount of the enzyme or enzyme mixture ranges from about 0.05% to about 2.0%.

12. A method in accordance with claim 1 wherein said method further comprises admixing the stool modulating composition with dry ingredients, feeding said composition to an extruder cooker and extruding said modulating composition to produce a dry bite size quadruped stool index modulatable food composition.

13. A method for increasing the digestibility of the diet of an animal which comprises preparing an aqueous meat fat slurry comprising fat and meat ingredients by homogenizing and emulsifying said ingredients in water to form a conditioned slurry, treating the conditioned slurry with an enzyme composition under conditions of pH and temperature for a time effective to cause a hydrolysis reaction resulting in the preparation of a food digestibility enhancing composition, and utilizing the food digestibility composition as an animal food or incorporating the food digestibility enhancing in an animal food in an amount effective to enhance the digestibility of the food.

14. A method in accordance with claim 13 which comprises an additional step of heating the food digestibility enhancing composition to a temperature and for a time effective to inactivate the enzyme following said hydrolysis reaction.

15. A method in accordance with claim 13 wherein said digestibility comprise increasing at least one of total protein, fat, ash, calorie and carbohydrate content of an animal food.

16. A pet food composition useful for modulating the stool index of droppings of an animal, said composition comprising an enzyme hydrolyzed meat fat composition in an amount effective to modulate the stool index of an animal.

17. A composition in accordance with claim 13 wherein said composition is a therapeutic amount.

18. A composition in accordance with claim 13 wherein said composition is a daily therapeutic dosage.

19. A composition in accordance with claim 17 wherein the enzyme is a protease.

20. A composition in accordance with claim 17 wherein the enzyme is lipase.

21. A composition in accordance with claim 17 wherein the stool index is increased and the degree of hydrolysis achieved is such that the weight average of the molecular weight distribution of the hydrolyzed slurry is in the range from about 5,000 Daltons to about 30,000 Daltons.

22. A composition in accordance with claim 17 wherein said animal is a dog.

23. A composition in accordance with claim 17 wherein the animal is a cat.

24. A method of preparing a pet food composition which comprises preparing an aqueous meat slurry comprising fat and protein by homogenizing and emulsifying the protein and fat in a meat slurry in water to form a conditioned slurry, treating the conditioned slurry with an enzyme composition under conditions of pH and temperature for a time effective to cause a reaction resulting in the production of a stool index modulating composition, utilizing the stool index modulating composition as an animal food or incorporating the stool index improving composition as a digest on or with an animal food in an amount effective to modulate the stool index of an animal.

25. A method in accordance with claim 24 which further comprises heating the stool index modulating composition to a temperature for a time effective to substantially inactivate the enzyme composition.

26. A method in accordance with claim 24 wherein said method further comprises selecting one or more meats from at least one of beef, lamb, chicken, pork, mutton, fish and poultry.

27. A method in accordance with claim 24 wherein said animal is a dog.

28. A method in accordance with claim 24 wherein the animal is a cat.

29. A method in accordance with claim 24 wherein modulating comprises increasing the firmness of animal stool droppings.

30. A method in accordance with claim 24 wherein the enzyme comprises a protease enzyme.

31. A method in accordance with claim 24 wherein said method further comprising admixing the modulating composition with dry ingredients, feeding modulating containing composition to an extruder cooker and extruding said modulating composition to produce a dry bite size animal stool index modulatable food composition.
